(19) United States
(12) Patent Application Publication
(10) Pub. No.: US 2024/0254395 A1
(43) Pub. Date: Aug. 1, 2024

(54) PYROLYSIS OF POLYURETHANE COMPOUND-CONTAINING MATERIAL IN ORDER TO RECOVER RAW MATERIALS

(71) Applicant: Covestro Deutschland AG, Leverkusen (DE)

(72) Inventors: Stefanie Eiden, Köln (DE); Rainer Bellinghausen, Odenthal (DE); Aurel Wolf, Wülfrath (DE); Christian Hahn, Leverkusen (DE); Tim Loddenkemper, Dormagen (DE); Carola Jendrzok, Bad Homburg (DE)

(21) Appl. No.: 18/562,385

(22) PCT Filed: Jun. 1, 2022
(86) PCT No.: PCT/EP2022/064853

§ 371 Date: Nov. 20, 2023

(30) Foreign Application Priority Data
Jun. 2, 2021 (EP) 21177501.0

(51) Int. Cl. C10B 53/07; B01J 21/04; B01J 23/02; C07C 209/62; C08G 18/32; C08G 18/76; C08J 11/12; C10B 57/06; C10B 57/14

(52) U.S. Cl. CPC C10B 53/07; B01J 21/04; B01J 23/02; C07C 209/62; C08G 18/324; C08G 18/7671; C08J 11/12; C10B 57/06; C10B 57/14; C08J 2375/04

(57) ABSTRACT

The invention relates to a process according to claim 1 and pyrolysis devices used therein for the pyrolysis of pyrolysis stock comprising polyurethane-containing material, allowing carrying out pyrolysis on an industrial scale. According to the invention, even with higher amounts of pyrolysis stock an amount of pyrolysis product is obtained that contains cleavage products which can be reused for the synthesis of polyurethane-containing material.

The invention relates to a pyrolysis process for the thermal utilization of polyurethane-containing material, to a corresponding use of a pyrolysis device, and to the product of the pyrolysis, which comprises raw materials of a kind that can be reused for polyurethane production.

Polyurethane-containing materials find use as cushioning or as insulation materials in the refrigeration and construction sectors. Polyurethane-containing foam materials in particular are used for example as cushioning elements, mattresses or insulation material. When using polyurethane-containing materials as insulation, for refrigerators or in construction for example, it is mainly rigid polyurethane foams that are used.

At the end of the service life of products containing polyurethane-containing material, they are replaced with new products and normally scrapped. The total amount of plastic waste that results increases every year. About 60% of the total amount is disposed of through incineration or landfill. When incinerated, CO₂ is emitted into the air, which contributes to global warming. Its low density means that plastic waste in landfills occupies a large volume and can from there also contribute to general pollution in rivers and seas. For this reason it is important to develop an efficient recycling method with which the waste problem can be solved and which at the same time allows fossil resources to be conserved. Since the abovementioned rigid polyurethane foams account for only a small part of the polymer market, at approximately 8%, the recycling of polyurethanes was not an initial focus of developments. However, the continuing growth of the plastics market means it is now important to develop recycling technologies for all polymer classes in order both to reduce CO₂ emissions and to conserve fossil energy sources.

The processes for recycling plastic waste can be roughly divided into three categories:
(1) mechanical recycling: Here, the plastic waste is melted down again as is and can then be reused. This is not possible for polyurethane elastomers.
(2) chemical and thermochemical recycling: Here, the plastic waste is depolymerized into monomers or broken down into smaller molecules in order to obtain useful chemical raw materials, and
(3) thermal recycling, in which the plastic waste is converted into offgas and heat energy.

The chemical raw materials obtained from thermochemical recycling can be used to synthesize new synthetic resins or other chemical products.

Thermochemical recycling is referred to as pyrolysis. In most cases pyrolysis is employed for packaging waste, affording a pyrolysis oil that is used as a kind of recycled naphtha in known refinery processes with crackers in the form of a drop-in solution. Little is known about the pyrolysis of polyurethanes.

The use of catalysts or additives in the pyrolysis process can lower operating temperatures, shorten reaction times, increase degradation efficiency, and restrict product distribution, making the process more efficient.

Kumagaia et al. (Journal of Analytical and Applied Pyrolysis 126 (2017) 337-345) describe a pyrolysis of flexible and rigid foams carried out at approx. 800° C. in which isocyanates, diamines, and a great many other fragments from the polyol are obtained.

M. Blazsó et al. (J. Chromatogr. A 1271 (2013) 217-220) showed that the use of a basic NH₄Y zeolite affords THF as the main product from the polyol. Two further easily identified products were aniline and 4-methylaniline, which form via the NH₄Y zeolites through the catalytic decomposition of MDI.

DE 2410505 C2 discloses a process for the thermal production of isocyanates from urethanes at 400 to 530° C. and reduced pressure. There was no mention of experiments with polyurethanes at normal pressure.

DE 2362915 proposes the hydrolysis of flexible polyurethane foams into diamines in a fluid bed.

None of the pyrolysis processes described in the prior art are suitable for industrial or commercial use, since they are mostly intended for use as pyrolyses on a microgram scale for analytical purposes. The pyrolyses presented in the prior art are therefore in each case carried out in pyrolysis devices that are not suitable for industrial or commercial use.

For the pyrolysis of larger amounts of polyurethane-containing materials in particular there is a need for novel, more selective processes with tailored catalysts and reactors, with which the formation of undesired by-products can be reduced. Polyurethane-containing materials should selectively afford pyrolysis products having a high content of cleavage products that can be reused for polyurethane synthesis, especially aromatic amine compounds such as aniline, toluidine, methylenedianiline (mMDA) or methylenedianiline polymers (pMDA).

The object was therefore to provide, for the commercial execution of a pyrolysis, a process and pyrolysis devices employable in said process for the pyrolysis of a pyrolysis feedstock that at least includes polyurethane-containing material, the use of which affords, even with relatively large amounts of pyrolysis feedstock, an amount of pyrolysis product that comprises cleavage products that can be reused for the synthesis of polyurethane-containing material, preferably in an amount of more than 40% by weight based on the total weight of polyurethane-containing material used.

In addition, a secondary object was to recover, to the greatest extent possible, aromatic amines containing structural units with alkylene-bridged aromatic units in the pyrolysis of polyurethane-containing material based on alkylene-bridged, aromatic isocyanates (for example methylenediphenylisocyanate (MDI) or methylendiphenylisocyanate polymers (pMDI)).

The present application therefore provides a pyrolysis process, comprising at least the following steps:
(a) introducing a pyrolysis feedstock at least comprising
   i) material comprising at least one polymeric compound having at least one polyurethane structural unit of the formula (I),

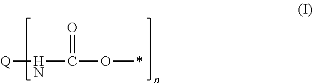

where
Q is a hydrocarbon radical having 8 to 70 carbon atoms, preferably 10 to 30 carbon atoms, and
n is a number from 2 to 10, preferably from 2 to 6, more preferably from 2 to 4,
and —* denotes a covalent bond to the polymer backbone, and
ii) at least one catalyst influencing the thermal degradation of said polymeric compound,
into a reactor;
(b) degradation of at least the material of the pyrolysis feedstock introduced in step (a) in the reactor at a temperature of 250° C. to 700° C. to obtain a gas-phase product as pyrolysate and a non-gas-phase pyrolysis residue, wherein
   (i) during said degradation, the amount of oxygen gas in the reactor is not more than 2.0% by volume based on the total volume of the gases present in the reactor, and
   (ii) during said degradation, the pyrolysate is discharged from the reactor, and
   (iii) said pyrolysis residue is discharged from the reactor;
(c) cooling the discharged pyrolysate to a temperature of less than 250° C. to obtain pyrolysis product selected from pyrolysate condensate, pyrolysate sublimate or a mixture thereof;
(d) optionally working up the pyrolysis product.

The term “pyrolysis feedstock” refers to the totality of the substances introduced into the reactor for the pyrolysis that therein undergo thermal treatment in the absence of oxygen gas or in the presence of a reduced amount of oxygen gas. The pyrolysis feedstock is preferably in solid form prior to its introduction into the reactor.

“Pyrolysate” is understood as meaning the totality of the products formed by pyrolysis that are in the gas phase in the reactor under the conditions of step (b) (more particularly in the form of a gas and/or as an aerosol).

“Pyrolysis residue” is understood as meaning the totality of the substances formed by pyrolysis and other residues of the pyrolysis feedstock that are not in the gas phase in the reactor under the conditions of step (b). Preference is given to the embodiments of the process characterized in that the pyrolysis residue in the reactor is solid.

“Pyrolysis product” is understood as meaning the totality of the products from the pyrolysate that in step (c) accumulate by condensation and/or resublimation when the pyrolysate cools. Pyrolysis product that is liquid is also referred to as pyrolysis oil.

Unless explicitly otherwise defined in this connection, a substance (for example material, pyrolysis feedstock, pyrolysate, pyrolysis product, pyrolysis residue) is “liquid” if it is in the liquid state at 20° C. and 1013 mbar. Unless explicitly otherwise defined in this connection, a substance (for example material, pyrolysis feedstock, pyrolysate, pyrolysis product, pyrolysis residue) is “solid” if it is in the solid state at 20° C. and 1013 mbar. Unless explicitly otherwise defined in this connection, a substance (for example material, pyrolysis feedstock, pyrolysate, pyrolysis residue) is “gaseous” if it is present as a gas at 20° C. and 1013 mbar.

A substance is “organic” if its chemical structure includes at least one covalent carbon-hydrogen bond.

In this application, the average molar masses specified for polymers or for polymeric ingredients are unless explicitly otherwise stated always weight-average molar masses Mw, which can in principle be determined by gel-permeation chromatography using an RI detector, it being expedient to perform the measurement against an external standard.

A “reactor” is a volume in which a chemical transformation, for example a thermal degradation of material from the pyrolysis feedstock, takes place. For a thermal degradation this can for example be the volume of a heated vessel in which the pyrolysis feedstock is contained.

It is in accordance with the invention advantageous when the pyrolysis feedstock is in accordance with the process of the invention introduced into a reactor characterized in that it is selected from a continuous stirred-tank reactor (CSTR), fixed-bed reactor, fluid-bed reactor, screw reactor, screw-conveyor reactor, entrained-flow reactor, entrainment-flow reactor, rotary-tube reactor, fluid-bed reactor, and drum reactor. More particularly, suitable reactors are preferably ones in which the pyrolysis feedstock can be introduced continuously and are selected in particular from a rotary-tube reactor, continuous stirred-tank reactor (CSTR), fixed-bed reactor (especially with continuous bed exchange (shaft reactor) with internal heat exchangers, preferably with internal heat exchanger tubes), screw reactor, screw-conveyor reactor, entrained-flow reactor, rotary-tube reactor or fluidized-bed reactor. In one embodiment of the process a very particularly preferred reactor is selected from a screw reactor, a rotary oven or fluidized bed. Further reactors preferred for the process according to the invention and embodiments thereof are described in the embodiments of the pyrolysis device of the invention and in an embodiment of the process with the use of a catalyst (vide infra).

According to the invention, the pyrolysis feedstock introduced into the reactor includes at least one material comprising at least one polymeric compound having at least one polyurethane structural unit of the formula (I),

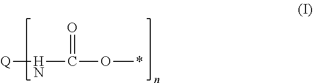

where
Q is a hydrocarbon radical having 8 to 70 carbon atoms, preferably 10 to 30 carbon atoms, and
n is a number from 2 to 10, preferably from 2 to 6, more preferably from 2 to 4, and —* denotes a covalent bond to the polymer backbone.

Q is preferably derived from aliphatic hydrocarbon units, cycloaliphatic hydrocarbon units, araliphatic hydrocarbon units, aromatic hydrocarbon units or heterocyclic hydrocarbon units. The process of the invention is particularly well suited to materials that comprise a polymeric compound of the aforementioned kind in which a group Q according to formula (I) includes at least one aromatic radical. Very particular preference as the structural unit of the formula (I) is given to ones in which the group Q contains at least two alkylene-bridged aromatic radicals, preferably at least two alkylene-bridged phenyl radicals, especially at least two methylene-bridged phenyl radicals.

A very particularly preferred embodiment of the process of the invention is characterized in that the polymeric compound contains, as at least one structural unit of the above formula (I), at least one polyurethane structural unit of the formula (Ia)

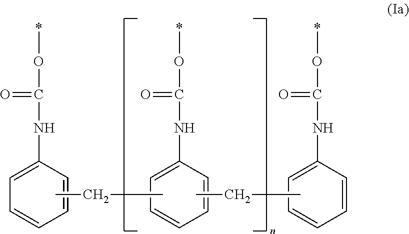

where
n is a number from 0 to 8, especially from 0 to 4, more preferably from 0 to 2, and —* denotes a covalent bond to the polymer backbone.

The polymeric compound present in said material can preferably be obtained by reaction at least of
i1) at least one organic isocyanate compound containing at least two to ten, especially two to eight, more preferably two to four, isocyanate groups attached to a hydrocarbon unit having 8 to 70 carbon atoms, preferably having 10 to 30 carbon atoms; with
i2) at least one organic compound having at least two hydroxy groups.

It is in a further embodiment preferable when the at least one organic isocyanate compound contains, as said hydrocarbon unit, a unit that has the number of carbon atoms stated in i1) and is derived from aliphatic hydrocarbon units, cycloaliphatic hydrocarbon units, araliphatic hydrocarbon units, aromatic hydrocarbon units or heterocyclic hydrocarbon units.

Particularly preferably, at least one compound corresponding to formula (II) is selected as said organic isocyanate compound

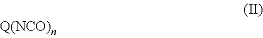

where n is a number from 2 to 4, preferably from 2 to 3, and Q is a radical selected from an aliphatic hydrocarbon radical having 8 to 70 carbon atoms, preferably 10 to 30 carbon atoms, a cycloaliphatic hydrocarbon radical having 8 to 70 carbon atoms, preferably 10 to 30 carbon atoms, an aromatic hydrocarbon radical having 8 to 70 carbon atoms, preferably 10 to 30 carbon atoms or an araliphatic hydrocarbon radical having 8 to 70 carbon atoms, preferably 10 to 30 carbon atoms.

When said polymeric compound contains at least one structural unit of the above formula (Ia) embodiment, a further preferred embodiment of the process has been found to be when at least one polyphenylpolymethylene polyisocyanate of the formula (III) is selected as the at least one organic polyisocyanate compound in step i1),

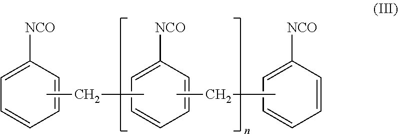

where n is a number from 0 to 8, especially from 0 to 4, more preferably from 0 to 2,

A preferred embodiment of the above step i2) is when at least one organic compound having at least two hydroxy groups is selected from polyester polyol, polyether polyol, polycarbonate polyol, polyetherester polyol, polyacrylate polyol, polyester polyacrylate polyol or mixtures thereof.

Preferably, at least one organic compound having at least two hydroxy groups is selected from the group comprising polyether polyols and/or polyester polyols.

The OH value of the employed polyol(s) may be for example >100 mg KOH/g to <800 mg KOH/g and the average OH functionality of the of the employed polyol(s) is >2. In the case of a single added polyol the OH value indicates the OH value of said polyol. In the case of mixtures, the average OH value is stated. This value may be determined in accordance with DIN 53240. The average OH functionality of the polyols is for example within a range from >2 to <6.

Employable polyether polyols include for example the polytetramethylene glycol polyethers obtainable through polymerization of tetrahydrofuran by cationic ring opening. Suitable polyether polyols also include addition products of styrene oxide, ethylene oxide, propylene oxide, butylene oxides and/or epichlorohydrin to di- or polyfunctional starter molecules. It is usual to employ polyether polyols with ethylene oxide or propylene oxide as chain extenders.

Examples of suitable starter molecules are ethylene glycol, diethylene glycol, butyl diglycol, glycerol, diethylene glycol, trimethylolpropane, propylene glycol, pentaerythritol, sorbitol, sucrose, ethylenediamine, toluenediamine, triethanolamine, butane-1,4-diol, hexane-1,6-diol and low-molecular-weight hydroxyl-containing esters of such polyols with dicarboxylic acids.

Employable polyester polyols include inter alia polycondensates of di- and also tri- and tetraols and di- and also tri- and tetracarboxylic acids or hydroxycarboxylic acids or lactones. Instead of the free polycarboxylic acids it is also possible to use the corresponding polycarboxylic anhydrides or corresponding polycarboxylic esters of lower alcohols for production of the polyesters. Examples of suitable diols are ethylene glycol, butylene glycol, diethylene glycol, triethylene glycol, polyalkylene glycols such as polyethylene glycol, and also propane-1,2-diol, propane-1,3-diol, butane-1,3-diol, butane-1,3-diol, hexane-1,6-diol and isomers, neopentyl glycol or neopentyl glycol hydroxypivalate. In addition, it is also possible to use polyols such as trimethylolpropane, glycerol, erythritol, pentaerythritol, trimethylolbenzene or tris(hydroxyethyl) isocyanurate.

Examples of polycarboxylic acids that may be used include phthalic acid, isophthalic acid, terephthalic acid, tetrahydrophthalic acid, hexahydrophthalic acid, cyclohexanedicarboxylic acid, adipic acid, azelaic acid, sebacic acid, glutaric acid, tetrachlorophthalic acid, maleic acid, fumaric acid, itaconic acid, malonic acid, suberic acid, succinic acid, 2-methylsuccinic acid, 3,3-diethylglutaric acid. 2,2-dimethylsuccinic acid, dodecanedioic acid, endomethylenetetrahydrophthalic acid, dimer fatty acid, trimer fatty acid, citric acid or trimellitic acid. It is also possible to use the corresponding anhydrides as the acid source.

If the average functionality of the polyol to be esterified is >2, it is additionally also possible to use monocarboxylic acids such as benzoic acid and hexanecarboxylic acid too.

Examples of hydroxycarboxylic acids that may be used as co-reactants for the production of a polyester polyol having terminal hydroxyl groups include hydroxy caproic acid, hydroxy butyric acid, hydroxydecanoic acid, hydroxystearic acid, and the like. Suitable lactones include caprolactone, butyrolactone and homologs. Employable polycarbonate polyols include hydroxyl-containing polycarbonates, for example polycarbonate diols. These are obtainable by reaction of carbonic acid derivatives, such as diphenyl carbonate, dimethyl carbonate or phosgene, with polyols, preferably diols, or from carbon dioxide. Examples of such diols are ethylene glycol, propane-1,2-diol and -1,3-diol, butane-1,3-diol and -1,4-diol, hexane-1,6-diol, octane-1,8-diol, neopentyl glycol, 1,4-bishydroxymethylcyclohexane, 2-methylpropane-1,3-diol, 2,2,4-trimethylpentane-1,3-diol, dipropylene glycol, polypropylene glycols, dibutylene glycol, poly butylene glycols, bisphenol A, and lactone-modified diols of the aforementioned type. Polyether polycarbonate diols may also be employed instead of or in addition to pure polycarbonate diols.

Employable polyetherester polyols are compounds containing ether groups, ester groups, and OH groups. Organic dicarboxylic acids having up to 12 carbon atoms are suitable for producing the polyetherester polyols, preferably aliphatic dicarboxylic acids having >4 to <6 carbon atoms or aromatic dicarboxylic acids used singly or in a mixture. Examples include suberic acid, azelaic acid, decanedicarboxylic acid, maleic acid, malonic acid, phthalic acid, pimelic acid and sebacic acid and in particular glutaric acid, fumaric acid, succinic acid, adipic acid, phthalic acid, terephthalic acid, and isoterephthalic acid. Derivatives of these acids that may be used include for example the anhydrides thereof and also the esters and monoesters thereof with low-molecular-weight monofunctional alcohols having >1 to <4 carbon atoms.

Preferably employed as the at least one organic compound having at least two hydroxy groups is preferably at least one aliphatic polyester polyol that in addition to structural units derived from adipic acid also contains structural units derived from glutaric acid, succinic acid and/or phthalic acid, preferably glutaric acid and/or succinic acid.

In addition to the structural unit of the formula (I) or (Ia) defined above, the polymeric compound of the material may optionally additionally contain an isocyanurate structural unit of the following formula

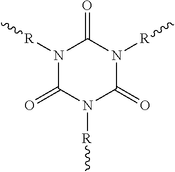

where R is a divalent hydrocarbon radical, especially a divalent aromatic hydrocarbon radical. For this embodiment it has been found to be preferable when the proportion of this isocyanurate structural unit amounts to not more than 40% by weight of the total weight of the material.

In one embodiment of the process according to the invention, said pyrolysis feedstock contains said polyurethane polymer-containing material in a total amount of 10.0% to 80.0% by weight, more preferably 30.0% to 70.0% by weight, in each case based on the total weight of the pyrolysis feedstock.

The material comprising said polymeric compound is preferably a foam, more preferably a polyurethane foam. When the material is present in the form of a polyurethane foam, it is in turn preferably a flexible polyurethane foam or a rigid polyurethane foam. A rigid polyurethane foam is a very particularly preferred embodiment of the material introduced in the pyrolysis feedstock in step a) of the process.

When said material is present in the form of a rigid polyurethane foam, it has preferably been found to be advantageous when the rigid polyurethane foam has in accordance with DIN 7726: 1982-05, at a compressive load for 10% compression, a compressive stress of >15 kPa, measured according to DIN 53421.

It has been found to be advantageous when said material of the pyrolysis feedstock is preferably introduced into the reactor in the form of solid particles (especially in the form of a granular mixture). A granular mixture of said material is formed from a multitude of loose, solid particles of said material, which in turn comprise what are known as grains. A grain is a term for the particulate constituents of powders (grains are the loose, solid particles), dusts (grains are the loose, solid particles), granules (loose, solid particles are agglomerates of several grains), and other granular mixtures. The flowability of a granular mixture relates to its ability to flow freely under its own weight from a pour test funnel having an outlet 16.5 mm in diameter.

The solid particles, more particularly the loose, solid particles of the granular mixture of said material introduced into the reactor, preferably have a median diameter X_{50.3} (volume average) of from 0.01 mm to 5 cm, preferably from 0.1 mm to 5 cm. The median particle size diameter X_{50.3} is determined by sieving or using a Camsizer particle size analyzer from Retsch.

The metering and processibility of the pyrolysis feedstock in the pyrolysis of the process of the invention can be simplified by said pyrolysis feedstock in step (a) also comprising in addition to said material at least one filler. Said filler preferably does not catalyze the thermal degradation of polyurethane during the pyrolysis. It is in turn particularly preferable when the filler is at least one metal oxide not catalytically active in the pyrolysis in the thermal degradation of polyurethane that is preferably selected from SiO₂.

Said material is preferably mixed with a filler, for example sand, thereby making continuous process control of the process according to the invention simpler. In particular, sticking of said material in the reactor and in the supply of the metering device to the reactor is avoided. In one embodiment, the filler and said material are supplied to the pyrolysis feedstock as a mixture in a volume ratio of filler to said material of at least 0.1:1 to 10:1.

The presence of at least one catalyst in the pyrolysis feedstock is mandatory. This catalyst influences the degradation reaction of said material. A suitable catalyst can lower the pyrolysis temperature, reduce the spectrum of products to the desired products and optionally minimize carbonization. For an efficient process, inexpensive catalysts are preferred.

Effective catalysts can be naturally occurring materials such as inorganic salts, refractory oxides, minerals, and industrial stones into which ions can optionally be exchanged in a simple ion-exchange process, since they do not require extensive synthesis. They are readily available and therefore relatively cheap. Synthetic catalysts such as zeolites (ZSM-5, zeolite X, Y, etc.) are on the other hand an example of catalysts that, although effective, are not cheap, since they have to be specially manufactured.

In addition to the function as a catalyst, catalysts can also simplify the metering and processibility of the pyrolysis feedstock in the pyrolysis. When using a catalyst, the amount of filler used can in this case be reduced. In one embodiment, the total amount of filler and catalyst is supplied to the pyrolysis feedstock as a mixture in a ratio to the amount of said material in a volume ratio of at least 0.1:1 to 10:1.

As a suitable said catalyst that is particularly preferred, the pyrolysis feedstock may comprise at least one basic catalyst. In the case of a solid basic catalyst, the number and strength of the basic centers in the catalyst can be determined by Fourier transform infrared spectroscopy and temperature-programmed desorption of CO₂ (TPD-CO₂), or determined using standard titrimetric methods.

In the pyrolysis reaction, preferably at least one catalyst is selected from the group consisting of alkaline inorganic materials, more preferably from the group of naturally occurring materials defined above. These inorganic materials may be refractory oxides. Refractory oxides are metal oxides that are stable at high temperatures from 300° C. to 700° C. Such oxides that function as catalysts include the oxides of aluminum, magnesium, zirconium, titanium, chromium, zinc, tin, and other metals or combinations of aluminum oxide with magnesium oxide and/or with calcium oxide.

Crystalline inorganic materials include aluminosilicates, silicon aluminum phosphates, silicalite, spinels, and other natural zeolites and clays. Especially suitable as said catalyst is thus at least one compound from the group consisting of inorganic salts, minerals, metal oxides, mixed oxides, clays, and zeolites.

Particularly preferably, the catalyst comprises at least one oxide of aluminum in the form of an oxide or mixed oxide and is present in a spinel structure, hydrotalcite structure or γ-Al₂O₃ structure.

A suitable catalyst is very particularly preferably a mixed oxide of Al₂O₃ and MgO. This is in turn very particularly preferably present in a spinel structure or hydrotalcite structure.

It has been found, for example, that when the catalyst is an Al₂O₃ mixed oxide and is present in a hydrocalcite structure, a higher proportion of mMDA is present in the pyrolysis product from the pyrolysis of MDI and/or pMDI. This catalyst thus allows a higher proportion of two-ring aromatic amine to be recovered.

It is possible to use both homogeneous and heterogeneous catalysts. However, preferred embodiments of the process according to the invention are those in which the catalyst is a heterogeneous catalyst. It has been found to be advantageous when the catalyst is introduced into the reactor preferably in the form of solid particles (especially in the form of a granular mixture). The heterogeneous catalyst used particularly preferably has a median particle size in the solid particles thereof, more particularly in the loose, solid particles of the granular mixture thereof, a median diameter X_{50.3} (volume average) of from 0.01 mm to 5 cm, preferably from 0.1 mm to 5 cm. The median particle size diameter X_{50.3} is determined by sieving or using a Camsizer particle size analyzer from Retsch.

When using a heterogeneous catalyst, it has in a further preferred embodiment been found to be particularly suitable when the catalyst particles have a smaller or the same particle size as the particles of said material. It is therefore preferable when the median particle size of the catalyst present in the pyrolysis feedstock corresponds at most to the median particle size of said material present therein.

If a catalyst is added, it may be admixed with the filler, coated onto the filler, or used as a substitute for the filler.

Since the catalyst used in the process according to the invention tends to form deposits of carbonized material and other carbon residues, when using a catalyst it is preferable to use a reactor that readily and preferably allows the catalyst to be discharged and continuously regenerated. Therefore, the use of a continuous stirred-tank reactor (CSTR), moving bed, screw reactor, screw-conveyor reactor, entrained-flow reactor, rotating-cone reactor, or fluidized-bed reactor is preferred over the use of a fixed-bed reactor.

The deactivated catalyst (deactivated for example through carbonization) is present in the pyrolysis residue and, after discharge of the pyrolysis residue from the reactor, can be supplied to a regenerator and after regeneration added to the pyrolysis feedstock of step (b) to be introduced into the pyrolysis reactor. Reactors that permit a short contact time, intensive mixing of the components in the feedstream with the catalyst, and for continuous recycling of the regenerated catalyst to the pyrolysis zone are most preferred. Since this is the case with a screw reactor, rotary oven or fluidized bed, these reactors are particularly preferred.

The pyrolysis feedstock introduced into the reactor in step (a) is at least partially broken down according to step (b), with the formation of pyrolysate and pyrolysis residue.

After being introduced into the reactor, the pyrolysis feedstock is heated to a temperature in the range from 250° C. to 700° C. A particularly successful improvement of the result of the process according to the invention can be achieved when, in one embodiment, the introduced pyrolysis feedstock is temperature-controlled at 250° C. to 700° C. and, on reaching this target temperature, the residence time of the correspondingly temperature-controlled pyrolysis feedstock until the time of discharge of the pyrolysis residue resulting therefrom is from 1 second to 2 hours, preferably between 2 minutes and 60 minutes, the temperature and the content of oxygen gas in the reactor during this time being the values defined in step (b).

When the pyrolysis feedstock in the process of the invention includes a catalyst, this catalyst is present in the pyrolysis residue that is discharged. In a further embodiment of the process, the discharged pyrolysis residue as described above is therefore supplied to a step for regeneration of the catalyst contained therein.

Independently thereof, good results can be achieved when the discharge of the pyrolysate from the reactor that takes place in step (b) is ensured by a gas stream passed through the reactor or by suction, and more preferably by the residence time of the pyrolysate, as the period between the time of introduction of said material introduced into the reactor in step (a) and the time of discharge of the pyrolysate, being from 0.1 seconds to 600 seconds, preferably between 0.5 seconds and 300 seconds, more preferably 0.5 seconds to 200 seconds.

If a gas stream is passed through the reactor for this purpose, an inert gas preferably selected from nitrogen, argon, CO₂, NO or a mixture thereof is particularly suitable as the gas for this gas stream.

If a gas stream is used to discharge the pyrolysate, it is preferable according to the invention when the flow rate of the gas stream in the reactor, as the superficial velocity, is in the range from 0.01 m/s to 20 m/s. If a fixed-bed reactor is chosen as the reactor, it is preferable according to the invention when the flow rate of the gas stream in the reactor, as the superficial velocity, is in the range from 0.03 m/s to 1 m/s. If a fluidized-bed reactor is chosen as the reactor, it is preferable according to the invention the flow rate of the gas stream in the reactor, as the superficial velocity, is in the range from 0.5 m/s to 2 m/s. If an entrained-flow reactor is chosen as the reactor, it is preferable according to the invention the flow rate of the gas stream in the reactor, as the superficial velocity, is in the range from 5 m/s to 20 m/s.

As the conditions of the invention for the degradation in the pyrolysis in step (b), the temperature in the reactor is 250 to 700° C. and the amount of oxygen gas in the reactor is from 0% to 2.0% by volume based on the total volume of the gases present in the reactor.

The amount of oxygen gas according to the invention is established by filling the reactor packed with said material of said pyrolysis feedstock with inert gas, in particular with nitrogen, argon, CO₂, NO or a mixture thereof. The inert gas may additionally be admixed with reactive gases other than oxygen gas, in particular gases selected from methane, gaseous H₂O, hydrogen gas or mixtures thereof.

To minimize the ingress of oxygen gas due to the introduction of the pyrolysis feedstock into the reactor, the pyrolysis feedstock can be freed from oxygen gas before it is introduced in step (a), for example by driving out the oxygen gas by stripping with a stripping gas, for example in a storage vessel upstream of the reactor. For example, an inert gas, more particularly nitrogen, argon, CO₂, NO or mixtures thereof, could in the storage vessel be passed as a stripping gas from above or from below (preferably from above) via a frit into the vessel and into the pyrolysis feedstock so as to drive out the oxygen gas.

In a further preferred embodiment of the process, the absolute pressure in step (b) is not more than 1.2 bar.

In a preferred embodiment of the process according to the invention, the temperature in step (b) is from 300° C. to 700° C., preferably from 400° C. to 600° C.

In a further preferred embodiment of the process according to the invention, the amount of oxygen gas in the reactor in step (b) is not more than 0.5% by volume, preferably not more than 0.1% by volume, in each case based on the total volume of the gases present in the reactor.

In a very preferred embodiment of the process according to the invention, firstly the temperature in step (b) is from 300° C. to 700° C., more preferably from 400° C. to 600° C., and secondly the amount of oxygen gas in the reactor is not more than 0.5% by volume, preferably not more than 0.1% by volume, in each case based on the total volume of the gases present in the reactor. It is in turn exceptionally preferable when, in addition, thirdly the absolute pressure in step (b) is not more than 1.2 bar.

A preferred embodiment of the process provides continuous process control. For this, at least steps (a) and (b) run concomitantly in the context of continuous process control.

The pyrolysis product obtained according to step (c) can be worked up using standard separation methods, for example distillation or selective condensation, thereby affording (i).

The process of the invention can be carried out with the aid of a suitably configured pyrolysis device. The invention therefore further provides for the use of a pyrolysis device comprising at least one metering device for feeding in pyrolysis feedstock, at least one heatable reactor for the pyrolysis, and at least one pyrolysate collector, wherein
said heatable reactor for the pyrolysis includes at least one heating element that can be used for temperature control of the reactor at a temperature of 250° C. to 700° C. and also at least one inlet for pyrolysis feedstock and at least one separate outlet for pyrolysate; and that
the metering device and heatable reactor for the pyrolysis are arranged and configured in relation to one another such that the metering device is connected via at least one supply line to an inlet for the pyrolysis feedstock of said reactor; and that
the heatable reactor for the pyrolysis and the pyrolysate collector are in fluid communication with one another such that it is possible for the preferably gaseous pyrolysate to be discharged from said pyrolysate outlet and for the discharged pyrolysate to be introduced into the pyrolysate collector; and that
at least one pyrolysate collector includes at least one cooling device, temperature-controlled at a temperature below 250° C., that in said collector can be used to lower the temperature of the pyrolysate discharged from said reactor to less than 250° C., with the formation of a pyrolysis product selected from pyrolysate condensate, pyrolysate sublimate or a mixture thereof, and including at least one container for collecting and discharging the pyrolysis product obtained by cooling; and that
at least one metering device for feeding in pyrolysis feedstock, at least one heatable reactor for the pyrolysis, and at least one pyrolysate collector are arranged and configured in relation to one another such that they can be operated concomitantly,
for obtaining an organic compound having at least one amino group through pyrolysis of polymeric compounds having at least one polyurethane structural unit of the formula (I),

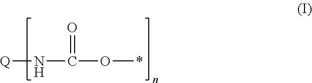

where
Q is a hydrocarbon radical having 8 to 70 carbon atoms and
n is a number from 2 to 10, preferably from 2 to 6, more preferably from 2 to 4, and —* denotes a covalent bond to the polymer backbone.

The polymeric compounds mentioned in the description of the process are also considered to be preferred polymeric compounds for the pyrolysis.

It is in accordance with the invention preferable to use said pyrolysis device for the recovery of at least one aromatic amino compound having at least one amino group, selected in particular from aniline, toluidine, methylenedianiline (mMDA), polymeric methylenedianiline (pMDA) or mixtures thereof.

The heating unit used for the heatable reactor of said pyrolysis device may for example be a heating element, for example a heating coil or heating plates, or a device for heating a gas stream and for introducing the heated gas stream into the reactor.

In a preferred embodiment, the reactor additionally includes at least one connection to a gas source with which a gas stream in the reactor, preferably having a flow rate, as a superficial velocity, of between 0.01 m/s and 20 m/s, flows via a regulator, for example a valve, through the reactor into the pyrolysate collector. If a fixed-bed reactor is chosen as the reactor, it is preferable according to the invention when the flow rate of the gas stream in the reactor, as the superficial velocity, is in the range from 0.03 m/s to 1 m/s. If a fluidized-bed reactor is chosen as the reactor, it is preferable according to the invention the flow rate of the gas stream in the reactor, as the superficial velocity, is in the range from 0.5 m/s to 2 m/s. If an entrained-flow reactor is chosen as the reactor, it is preferable according to the invention the flow rate of the gas stream in the reactor, as the superficial velocity, is in the range from 5 m/s to 20 m/s.

The gas stream from the gas source can for example be heated by the heating unit before it is introduced into the reactor as described above.

The pyrolysate collector of the pyrolysis device preferably comprises a cooling device that in said collector can be used to lower the temperature of the pyrolysate discharged from said reactor to less than 50° C. (more preferably to less than 30° C.), with the formation of a pyrolysis product. Cooling units that work according to the heat exchanger principle are particularly suitable for this. The pyrolysate collector can here be fitted out as a selective condenser for a selective condensation of pyrolysis product constituents present in the pyrolysate.

A “fluid connection” is in accordance with the invention understood as meaning a part of the device that connects parts of the system to one another and through which a substance that may be in any state of matter can be transported from one plant component to the next, for example a supply line in the form of a pipe.

The process according to the invention and the device according to the invention contribute to achieving the object set out above and in step (c) of the process provide as the pyrolysis product a composition at least comprising, in each case based on the total weight of the composition,
(i) from 0% and 40% by weight of a total amount of at least one aromatic compound having at least two amino groups,
(ii) between 0% and 35% by weight of a total amount of at least one aromatic compound having just one amino group, preferably aniline,
(iii) from 0% to 40% by weight of a total amount of at least one hydrocarbon compound containing at least one functional group having at least one oxygen atom and no functional group having a nitrogen atom,
(iv) where the proportions by weight within the parts by weight ranges from (i), (ii) and (iii) are selected such that the sum total of the selected proportions by weight from (i), (ii) and (iii) together with the proportions by weight of other ingredients add up to 100% by weight.

In a preferred embodiment, the composition under (i) contains from 0% to 40% by weight of a compound of the formula (IV)

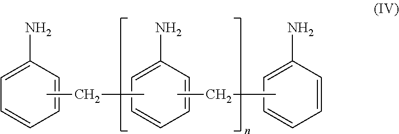

where n is a number from 0 to 8, especially from 0 to 4, more preferably from 0 to 2,

Further preferred pyrolysis products comprise more (i) aromatic compounds having at least two amino groups than (ii) aromatic compounds having just one amino group.

In a preferred embodiment, the composition under (ii) contains between 0% and 35% by weight of a total amount of aromatic amino compound having at least one amino group, selected from aniline, toluidine or mixtures thereof.

It is in another preferred embodiment in addition advantageous when the pyrolysis product contains between 0% and 40% by weight of a total amount of at least one aromatic compound having at least two amino groups.

In a preferred embodiment, the composition under (iii) comprises between 0% and 40% by weight of hydrocarbon compounds containing at least one functional group having at least one oxygen atom and no functional group having a nitrogen atom. These hydrocarbon compounds are in turn particularly preferably selected from compounds having a molar mass of not more than 300 g/mol, especially not more than 250 g/mol. More preferably, said hydrocarbon compounds are selected from acetone, dimethyldioxane, propene, 1,2-propylene glycol, dipropylene glycol, tripropylene glycol or tetrapropylene glycol.

In summary but without limitation, the following aspects 1 to 30 of the invention are to be regarded as further embodiments, the features of which are to be interpreted in accordance with the above description of the invention:

1. A pyrolysis process, comprising at least the following steps:
(a) introducing a pyrolysis feedstock at least comprising
   i) material comprising at least one polymeric compound having at least one polyurethane structural unit of the formula (I),

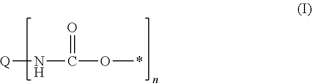

where
Q is a hydrocarbon radical having 8 to 70 carbon atoms, preferably 10 to 30 carbon atoms, and
n is a number from 2 to 10, preferably from 2 to 6, more preferably from 2 to 4,
and —* denotes a covalent bond to the polymer backbone, and
ii) at least one catalyst influencing the thermal degradation of said polymeric compound, into a reactor:
(b) degradation at least of the material of the pyrolysis feedstock introduced in step (a) in the reactor at a temperature of 250° C. to 700° C. to obtain a gas-phase product as pyrolysate and a non-gas-phase pyrolysis residue, wherein
   (i) during said degradation, the amount of oxygen gas in the reactor is not more than 2.0% by volume based on the total volume of the gases present in the reactor, and
   (ii) during said degradation, the pyrolysate is discharged from the reactor, and
   (iii) said pyrolysis residue is discharged from the reactor:
(c) cooling the discharged pyrolysate to a temperature of less than 250° C. to obtain pyrolysis product selected from pyrolysate condensate, pyrolysate sublimate or a mixture thereof;
(d) optionally working up the pyrolysis product.

2. The process according to aspect 1, characterized in that the reactor is selected from a continuous stirred-tank reactor (CSTR), fixed-bed reactor, fluid-bed reactor, screw reactor, screw-conveyor reactor, entrained-flow reactor, rotary-tube reactor, and drum reactor, in particular selected from continuous stirred-tank reactor (CSTR), fixed-bed reactor (especially with continuous bed exchange (shaft reactor) preferably with internal heat exchanger tubes), a screw reactor, a screw-conveyor reactor, an entrained-flow reactor, rotary-tube reactor or fluidized-bed reactor.

3. The process according to either of aspects 1 or 2, characterized in that the temperature in step (b) is from 300° C. to 700° C., preferably 400° C. to 600° C.

4. The process according to any of the preceding aspects, characterized in that the absolute pressure in step (b) is not more than 1.2 bar.

5. The process according to any of the preceding aspects, characterized in that the introduced pyrolysis feedstock is temperature-controlled at 250° C. to 700° C. and that, on reaching this target temperature, the residence time of the correspondingly temperature-controlled pyrolysis feedstock until the time of discharge of the pyrolysis residue resulting therefrom is from 1 second to 5 hours, preferably between 2 minutes and 120 minutes.

6. The process according to any of the preceding aspects, characterized in that the discharge of the pyrolysate from the reactor is ensured by a gas stream passed through the reactor or by suction, and preferably by the residence time of the pyrolysate, as the period between the time of formation of the pyrolysate and the time of discharge of the resulting pyrolysate from the reactor, being from 0.1 seconds to 600 seconds, preferably between 0.5 seconds and 300 seconds, more preferably 0.5 seconds to 200 seconds.

7. The process according to any of the preceding aspects, characterized in that the discharge of the pyrolysate from the reactor is ensured by a gas stream passed through the reactor, the flow rate of which in the reactor, as the superficial velocity, being in the range from 0.01 m/s to 20 m/s.

8. The process according to any of the preceding aspects, characterized in that the pyrolysis residue in the reactor is solid.

9. The process according to any of the preceding aspects, characterized in that at least steps (a) and (b) run concomitantly in the context of continuous process control.

10. The process according to any of the preceding aspects, characterized in that the amount of oxygen gas in the reactor in step (b) is not more than 0.5% by volume, preferably not more than 0.1% by volume, in each case based on the total volume of the gases present in the reactor.

11. The process according to any of the preceding aspects, characterized in that an inert gas, in particular nitrogen, argon, CO₂, NO or mixtures thereof, is passed through the reactor packed with said material.

12. The process according to aspect 11, characterized in that the inert gas may additionally be admixed with reactive gases other than oxygen gas, in particular gases selected from methane, gaseous H₂O, hydrogen gas or mixtures thereof.

13. The process according to any of the preceding aspects, characterized in that the polymeric compound of the polyurethane material additionally contains an isocyanurate structural unit of the following formula

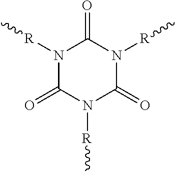

where R is a divalent hydrocarbon radical, especially a divalent aromatic hydrocarbon radical.

14. The process according to any of the preceding aspects, characterized in that the polymeric compound contains at least one polyurethane structural unit of the formula (Ia)

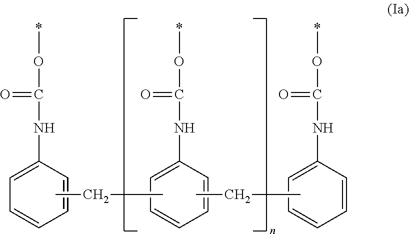

where
n is a number from 0 to 8, especially from 0 to 4, more preferably from 0 to 2, and —* denotes a covalent bond to the polymer backbone.

15. The process according to any of the preceding aspects, characterized in that the polymeric compound is obtained by reaction at least of
i1) at least one organic isocyanate compound containing at least two to ten, especially two to eight, more preferably two to four, isocyanate groups attached to a hydrocarbon unit having 8 to 70 carbon atoms, preferably having 10 to 30 carbon atoms; with
i2) at least one organic compound having at least two hydroxy groups.

16. The process according to aspect 15, characterized in that the at least one organic isocyanate compound contains, as said hydrocarbon unit, a unit that has the number of carbon atoms mentioned above and is derived from aliphatic hydrocarbon units, cycloaliphatic hydrocarbon units, araliphatic hydrocarbon units, aromatic hydrocarbon units or heterocyclic hydrocarbon units.

17. The process according to aspect 15 or 16, characterized in that at least one compound corresponding to formula (II) is selected as said organic isocyanate component

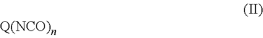

where n is a number from 2 to 4, preferably from 2 to 3, and Q is a radical selected from an aliphatic hydrocarbon radical having 8 to 70 carbon atoms, preferably having 10 to 30 carbon atoms, a cycloaliphatic hydrocarbon radical having 8 to 70 carbon atoms, preferably 10 to 30 carbon atoms, an aromatic hydrocarbon radical having 8 to 70 carbon atoms, preferably 10 to 30 carbon atoms or an araliphatic hydrocarbon radical having 8 to 70 carbon atoms, preferably 10 to 30 carbon atoms.

18. The process according to any of aspects 1 to 17, characterized in that, in the corresponding formula (I) and for aspect 18 in formula (II) too, Q contains at least two alkylene-bridged aromatic radicals, preferably at least two alkylene-bridged phenyl radicals, especially two methylene-bridged phenyl radicals.

19. The process according to any of aspects 15 to 18, characterized in that at least one polyphenylpolymethylene polyisocyanate of the formula (III) is selected as the organic polyisocyanate component,

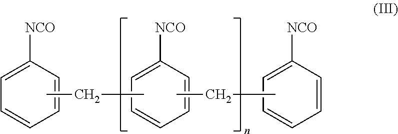

where n is a number from 0 to 8, especially from 0 to 4, more preferably from 0 to 2,

20. The process according to any of aspects 15 to 19, characterized in that at least one organic compound having at least two hydroxy groups is selected from polyester polyol, polyether polyol, polycarbonate polyol, polyetherester polyol, polyacrylate polyol, polyester polyacrylate polyol or mixtures thereof, preferably at least one aliphatic polyester polyol that in addition to structural units derived from adipic acid also contains structural units derived from glutaric acid, succinic acid and/or phthalic acid, preferably glutaric acid and/or succinic acid.

21. The process according to any of the preceding aspects, characterized in that the material is present in the pyrolysis feedstock as a rigid polyurethane foam, preferably as a rigid polyurethane foam that has in accordance with DIN 7726: 1982-05, at a compressive load for 10% compression, a compressive stress of >15 kPa, measured according to DIN 53421.

22. The process according to any of the preceding aspects, characterized in that said material is introduced into the reactor in the form of solid particles, especially in the form of a granular mixture.

23. The process according to aspect 22, characterized in that the solid particles, more particularly the loose, solid particles of the granular mixture of said material introduced into the reactor, have a median diameter X_{50.3} (volume average) of from 0.01 mm to 5 cm.

24. The process according to any of the preceding aspects, characterized in that the catalyst is at least one compound from the group consisting of inorganic salts, minerals, metal oxides, mixed oxides, clays, and zeolites, preferably a mixed oxide of Al₂O; and MgO.

25. The process according to any of the preceding aspects, characterized in that the catalyst is a basic catalyst.

26. The process according to any of the preceding aspects, characterized in that the catalyst is a heterogeneous catalyst.

27. The process according to any of the preceding aspects, characterized in that said pyrolysis feedstock contains said material in a total amount based on the total weight thereof of 10.0% to 80.0% by weight, more preferably 30.0% to 70.0% by weight.

28. The use of a pyrolysis device comprising at least one metering device for feeding in pyrolysis feedstock, at least one heatable reactor for the pyrolysis, and at least one pyrolysate collector, wherein
said heatable reactor for the pyrolysis includes at least one heating element that can be used for temperature control of the reactor at a temperature of 250° C. to 700° C. and also at least one inlet for pyrolysis feedstock and at least one separate outlet for pyrolysate; and that the metering device and heatable reactor for the pyrolysis are arranged and configured in relation to one another such that the metering device is connected via at least one supply line to an inlet for the pyrolysis feedstock of said reactor; and that
the heatable reactor for the pyrolysis and the pyrolysate collector are in fluid communication with one another such that it is possible for the preferably gaseous pyrolysate to be discharged from said pyrolysate outlet and for the discharged pyrolysate to be introduced into the pyrolysate collector; and that
at least one pyrolysate collector includes at least one cooling device, temperature-controlled at a temperature below 250° C., that in said collector can be used to lower the temperature of the pyrolysate discharged from said reactor to less than 250° C., with the formation of a pyrolysis product selected from pyrolysate condensate, pyrolysate sublimate or a mixture thereof, and including at least one container for collecting and discharging the pyrolysis product obtained by cooling; and that
at least one metering device for feeding in pyrolysis feedstock, at least one heatable reactor for the pyrolysis, and at least one pyrolysate collector are arranged and configured in relation to one another such that they can be operated concomitantly,
for obtaining an organic compound having at least one amino group through pyrolysis of polymeric compounds having at least one polyurethane structural unit of the formula (I),

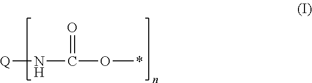

where
Q is a hydrocarbon radical having 8 to 70 carbon atoms and
n is a number from 2 to 10, preferably from 2 to 6, more preferably from 2 to 4, and —* denotes a covalent bond to the polymer backbone.

29. The use according to aspect 28, characterized in that the pyrolysis device is used for the recovery of at least one aromatic amino compound having at least one amino group, selected in particular from aniline, toluidine, methylenedianiline (mMDA), polymeric methylenedianiline (pMDA) or mixtures thereof.

30. A composition at least comprising, in each case based on the total weight of the composition,
(i) from 0% and 40% by weight of a total amount of at least one aromatic compound having at least two amino groups,
(ii) between 0% and 35% by weight of a total amount of at least one aromatic compound having just one amino group, preferably aniline, (iii) between 0% and 40% by weight of hydrocarbon compounds containing at least one functional group having at least one oxygen atom and no functional group having a nitrogen atom,
(iii) from 0% to 40% by weight of a total amount of at least one hydrocarbon compound containing at least one functional group having at least one oxygen atom and no functional group having a nitrogen atom,
(iv) where the proportions by weight within the parts by weight ranges from (i), (ii) and (iii) are selected such that the sum total of the selected proportions by weight from (i), (ii) and (iii) together with the parts by weight of other ingredients add up to 100% by weight.

### EXAMPLES

### a) Providing the Pyrolysis Feedstock:

A rigid polyurethane foam was produced by standard processes from the components shown in Table 1.

TABLE 1
Components of the rigid foam:

| | % by wt. |
|---|---|
| Constituents | |
| Polyol having an OH value of 450 | 30.10 |
| Polyol with o-tolylenediamine as starter | 8.60 |
| having an OH value of 400 | |
| Polyol having an OH value of 112 | 2.58 |
| Mixture of | 0.86 |
| Polycat ® 5, Polycat ® 8 and Polycat ® 41 ¹ | |
| Silicone (stabilizer) | 0.86 |
| Isocyanate | |
| Desmodur ® 44V20 (pMDI) | 57.00 |

¹ catalysts, sold by Evonik, Germany
² liquid, dark brown isocyanate from Covestro Deutschland AG, based on diphenylmethane 4,4′-diisocyanate (MDI) with isomers and higher-functional homologs (pMDI)

To provide the various pyrolysis feedstocks P1 to P3, 1 g of the finely grated rigid polyurethane foam was intimately mixed with 4 g of one of the catalyst powders listed in Table 2. Pyrolysis feedstock P4 contained, as a comparison, no catalyst.

TABLE 2
Catalyst C1 to C3 and pyrolysis feedstock P1 to P3

| | Catalyst: | | |
|---|---|---|---|
| | C1 | C2 | C3 |
| | in pyrolysis | | |
| | P1 | P2 | P3 |
| % by wt. MgO | 5.0 | 70.0 | 0.0 |
| % by wt. Al₂O₃ | 95.0 | 30.0 | 100.0 |
| Structure | Basic Al₂O₃ | Hydrotalcite | γ-Al₂O₃ |
| | precursor | | |
| Particle size d₅₀ | 40 | 40 | 40 |

### b) Pyrolysis Procedure

The pyrolysis of the rigid polyurethane foam was carried out at 500° C. in a fixed-bed reactor having a volume of 25 ml with a through-flow of N₂.

The flow rate of the nitrogen gas stream (superficial velocity) in the reactor was 0.07 m/s. A pyrolysis feedstock according to Table 1 was introduced into the reactor. The residence time of the introduced polyurethane material was 30 min. The reactor was heated to 500° C. and then held at this temperature for 30 min. Situated downstream of the reactor were three condensers for the separation of the liquid components from the resulting pyrolysis gas. The resulting content of carbonized material was determined by weighing the catalyst powder after the pyrolysis. The gas downstream of the three condensers was characterized by online IR. The components of the pyrolysis product obtained in the form of an oil were determined by GC-FID. This was done using an Agilent 7890A with a Supelco SPB 50 column. The pyrolysis oil was diluted 1:50 or 1:100 with acetone.

TABLE 3
Composition of the pyrolysate obtained [% by wt.]:

| | Pyrolysis feedstock | | | |
|---|---|---|---|---|
| | P1 | P2 | P3 | P4 |
| | Catalyst | | | |
| | C1 | C2 | C3 | without |
| Aniline* | 19.0 | 11.0 | 10.0 | — |
| Methylaniline* | 8.3 | 3.0 | 5.0 | — |
| mMDA* | 2.3 | 20.0 | — | 4.1 |
| Oil (not characterized) | 40.0 | 29.0 | 35.0 | 53.2 |
| containing pMDA* | | | | |
| Gas phase after condensation | | | | |
| H₂O | — | 0.5 | 20.0 | — |
| CO₂ | 10.0 | 13.0 | — | 10.0 |
| CO | — | 3.0 | — | — |
| Carbonized material | 10.0 | 10.0 | 30.0 | 17.3 |
| Other gases | 10.4 | 5.5 | — | 15.7 |
| Total | 100.0 | 100.0 | 100.0 | 100.0 |

*denotes constituents of the pyrolysis product

The pyrolysis product P2 containing the catalyst C2 was used to carry out a pyrolysis experiment at 500° C. and a pyrolysis experiment at 470° C. The distribution of the amines obtained was compared (see Table 4). This showed that at 470° C. more methylenedianiline (mMDA) is obtained in the pyrolysis product.

TABLE 4
Comparison of the distribution of the
aromatic amines obtained [% by wt.]:

| | P2 | P2 |
|---|---|---|
| Product | 500° C. | 470° C. |
| Anilines | 11 | 3 |
| Methylaniline | 3 | 3 |
| mMDA | 20 | 30 |

1. A pyrolysis process, comprising:
(a) introducing a pyrolysis feedstock comprising:
i) a material comprising at least one polymeric compound having at least one polyurethane structural unit of the formula (I), 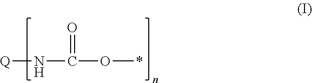
where
Q is a hydrocarbon radical having 8 to 70 carbon atoms, and
n is a number having a value of 2 to 10,
and —* denotes a covalent bond to the polymer backbone,
and
ii) at least one catalyst influencing thermal degradation of said polymeric compound, into a reactor;
(b) degradation at least of the material of the pyrolysis feedstock introduced in step (a) in the reactor at a temperature of 250° C. to 700° C. to obtain a gas-phase product as pyrolysate and a non-gas-phase pyrolysis residue, wherein
(i) during said degradation, the amount of oxygen gas in the reactor is not more than 2.0% by volume based on the total volume of the gases present in the reactor, and
(ii) during said degradation, the pyrolysate is discharged from the reactor, and
(iii) said pyrolysis residue is discharged from the reactor;
(c) cooling the discharged pyrolysate to a temperature of less than 250° C. to obtain a pyrolysis product selected from pyrolysate condensate, pyrolysate sublimate or a mixture thereof; and
(d) optionally working up the pyrolysis product.

2. The process as claimed in claim 1, wherein the temperature in step (b) is from 300° C. to 700° C.

3. The process as claimed in claim 1, wherein the absolute pressure in step (b) is not more than 1.2 bar.

4. The process as claimed in claim 1, wherein the introduced pyrolysis feedstock is temperature-controlled at a target temperature of 250° C. to 700° C. and that, on reaching the target temperature, the residence time of the temperature-controlled pyrolysis feedstock until the time of discharge of the pyrolysis residue resulting therefrom is from 1 second to 5 hours.

5. The process as claimed in claim 1, wherein the step of discharging the pyrolysate from the reactor comprises (i) passing a gas stream through the reactor or (ii) suction.

6. The process as claimed in claim 1, wherein the step of discharging the pyrolysate from the reactor comprises passing a gas stream through the reactor, the flow rate of which in the reactor, as the superficial velocity, is from 0.01 m/s to 20 m/s.

7. The process as claimed in claim 1, wherein at least steps (a) and (b) run concomitantly in the context of continuous process control.

8. The process as claimed in claim 1, wherein the amount of oxygen gas in the reactor in step (b) is not more than 0.5% by volume, based on the total volume of the gases present in the reactor.

9. The process as claimed in claim 1, wherein an inert gas is passed through the reactor packed with said material.

10. The process as claimed in claim 1, wherein the polymeric compound contains at least one polyurethane structural unit of the formula (Ia) 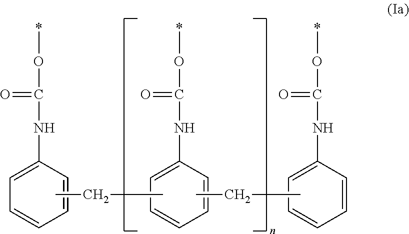
where
n is a number having a value of 0 to 8, and —* denotes a covalent bond to the polymer backbone.

11. The process as claimed in claim 1, wherein the polymeric compound is obtained by reaction at least of
i1) at least one organic isocyanate compound containing two to ten isocyanate groups attached to a hydrocarbon unit having 8 to 70 carbon atoms; with
i2) at least one organic compound having at least two hydroxy groups.

12. The process as claimed in claim 11, wherein the at least one organic isocyanate compound contains, as said hydrocarbon unit, a unit that is derived from aliphatic hydrocarbon units, cycloaliphatic hydrocarbon units, araliphatic hydrocarbon units, aromatic hydrocarbon units or heterocyclic hydrocarbon units.

13. The process as claimed in claim 11, wherein the at least one organic isocyanate component comprises at least one polyphenylpolymethylene polyisocyanate of the formula (III), 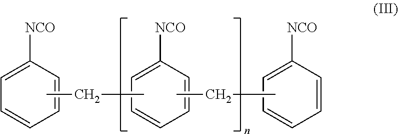
where n is a number having a value of 0 to 8.

14. The process as claimed in claim 1, wherein said material is introduced into the reactor in the form of solid particles.

15. The process as claimed in claim 1, wherein the catalyst comprises at least one compound from the group consisting of inorganic salts, minerals, metal oxides, mixed oxides, clays, and zeolites.

16. The process as claimed in claim 1, wherein the catalyst comprises a basic catalyst.

17. The process as claimed in claim 1, wherein the catalyst comprises a heterogeneous catalyst.

18. (canceled)

19. A composition comprising, in each case based on the total weight of the composition,
(i) 0% and 40% by weight of at least one aromatic compound having at least two amino groups,
(ii) 0% and 35% by weight of at least one aromatic compound having just one amino group,
(iii) 0% to 40% by weight of at least one hydrocarbon compound containing at least one functional group having at least one oxygen atom and no functional group having a nitrogen atom,
(iv) where the proportions by weight within the parts by weight ranges from (i), (ii) and (iii) are selected such that the sum total of the selected proportions by weight from (i), (ii) and (iii) together with the parts by weight of other ingredients add up to 100% by weight.
